(19) United States
(12) Patent Application Publication
(10) Pub. No.: US 2024/0148548 A1
(43) Pub. Date: May 9, 2024

(54) NOZZLE FOR A FLUID DELIVERY DEVICE

(71) Applicant: TWENTY TWENTY THERAPEUTICS LLC, South San Francisco, CA (US)

(72) Inventor: Timothy Stowe, Alameda, CA (US)

(21) Appl. No.: 18/387,004

(22) Filed: Nov. 3, 2023

Related U.S. Application Data
(60) Provisional application No. 63/422,468, filed on Nov. 4, 2022.

(51) Int. Cl. A61F 9/00

(52) U.S. Cl. CPC A61F 9/0008

(57) ABSTRACT

A nozzle for a fluid delivery device includes a nozzle head. The nozzle head defines an inner head chamber for a fluid. The nozzle head has at least one nozzle opening through which fluid is configured to be selectively delivered from the head chamber to a target site during use of the device. The nozzle head is at least partially formed from a deformable material. The nozzle head is selectively deflectable between an opened condition in which the at least one nozzle opening is at least partially open to permit fluid flow therethrough and a closed condition in which the at least one nozzle opening is at least partially closed to restrict fluid flow therethrough.

### RELATED APPLICATION

This application claims priority from U.S. Provisional Application No. 63/422,468, filed 4 Nov. 2022, the subject matter of which is incorporated herein by reference in its entirety.

### TECHNICAL FIELD

This disclosure generally relates to a nozzle for a fluid delivery device. This disclosure also generally relates to a fluid delivery device and a method of delivering fluid to a user's eye using the fluid delivery device.

### BACKGROUND

With conventional eye drop systems, preservatives are often included in the dispensed fluid to prevent the growth of bacterial or viral germs. While a filter may be used to at least partially prevent the preservative from reaching a user's eye, the filter may not be applicable to all types of fluids/formulations. Eye drop systems that do not include preservatives often include built-in barriers/filters and unidirectional valves. An example of such eye drop systems include multi-dose preservative-free (MDPF) bottles. U.S. Pat. No. 9,345,616, issued 24 May 2016 to Grevin at al. and titled “LIQUID DISPENSING DEVICE EQUIPPED WITH AN AIR DUCT”, the subject matter of which is incorporated herein by reference in its entirety, discloses a MDPF bottle having a unidirectional valve that prevents the backflow of fluid and bacteria into the MDPF bottle.

Certain MDPF bottles, however, may be difficult for certain users to use to administer a drop, as the squeeze force for ejecting a drop from those MDPF bottle may be greater than that required for non-MDPF bottles. Further, the structural design of conventional MDPF bottles provides only for a substantially “vertical” delivery of fluid. In certain cases, a differently angled—e.g., substantially “horizontal”—delivery of fluid may be more desirable.

### SUMMARY

In an aspect, alone or in combination with any other aspect, a nozzle for a fluid delivery device comprises a nozzle head. The nozzle head defines an inner head chamber for a fluid. The nozzle head has at least one nozzle opening through which fluid is configured to be selectively delivered from the head chamber to a target site during use of the device. The nozzle head is at least partially formed from a deformable material. The nozzle head is selectively deflectable between an opened condition in which the at least one nozzle opening is at least partially open to permit fluid flow therethrough and a closed condition in which the at least one nozzle opening is at least partially closed to restrict fluid flow therethrough.

In an aspect, alone or in combination with any other aspect, a fluid delivery device for delivering fluid to an eye of a user comprises a cartridge and an applicator. The cartridge includes a container and the nozzle coupled to the container. The container has an inner container chamber configured to accommodate a fluid. The head chamber is in fluid communication with the container chamber such that the fluid selectively flows from the container chamber to the head chamber. The applicator is configured to accommodate the cartridge. The applicator includes an actuator for selectively manipulating the nozzle head between the opened condition and the closed condition.

In an aspect, alone or in combination with any other aspect, a method of delivering fluid to a user's eye comprises providing the fluid delivery device. The cartridge is inserted into the applicator. An applicator opening of the applicator is aligned with the user's eye. The actuator is activated to manipulate the nozzle head from the opened condition to the closed condition. Fluid is delivered from the head chamber, through the nozzle and applicator openings, and to the user's eye as the nozzle head is manipulated from the opened condition to the closed condition.

### BRIEF DESCRIPTION OF THE DRAWINGS

For a better understanding, reference may be made to the accompanying drawings, in which:

FIG. 1 is a perspective front view of a nozzle for a fluid delivery device, including the nozzle in a first condition;

FIG. 2 is a front view of a component of the nozzle of FIG. 1;

FIG. 3 is a perspective front view of the nozzle of FIG. 1, including the nozzle in a second condition;

FIG. 4 is a front view of a component of the nozzle of FIG. 3;

FIG. 5 is an exploded view of a cartridge including the nozzle of FIG. 1; and

FIG. 6 is a schematic depiction of a fluid delivery device including the cartridge of FIG. 5.

### DESCRIPTION OF ASPECTS OF THE DISCLOSURE

As used herein, the term “user” can be used interchangeably to refer to an individual who prepares for, assists with, and/or performs the operation of a tool, and/or to an individual who prepares for, assists with, and/or performs a procedure.

As used herein, the singular forms “a,” “an” and “the” can include the plural forms as well, unless the context clearly indicates otherwise. It will be further understood that the terms “comprises” and/or “comprising,” as used herein, can specify the presence of stated features, steps, operations, elements, and/or components, but do not preclude the presence or addition of one or more other features, steps, operations, elements, components, and/or groups thereof.

As used herein, the term “and/or” can include any and all combinations of one or more of the associated listed items.

As used herein, phrases such as “between X and Y” can be interpreted to include X and Y.

It will be understood that when an element is referred to as being “on,” “connected” to, “coupled” with, etc., another element, it can be directly on, connected to or coupled with the other element or intervening elements may also be present. It will also be appreciated by those of skill in the art that references to a structure or feature that is disposed “adjacent” another feature may not have portions that overlap or underlie the adjacent feature.

It will be understood that, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or steps) is not limited to the order presented in the claims or Figures unless specifically indicated otherwise.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from about 1 to about 6 should be considered to have specifically disclosed subranges such as from 1 to 3, from 1 to 4, from 1 to 5, from 2 to 4, from 2 to 6, from 3 to 6, etc., as well as individual and partial numbers within that range, for example, 1, 2, 3, 4, 5, 5.5 and 6. This applies regardless of the breadth of the range.

The invention comprises, consists of, or consists essentially of the following features, in any combination.

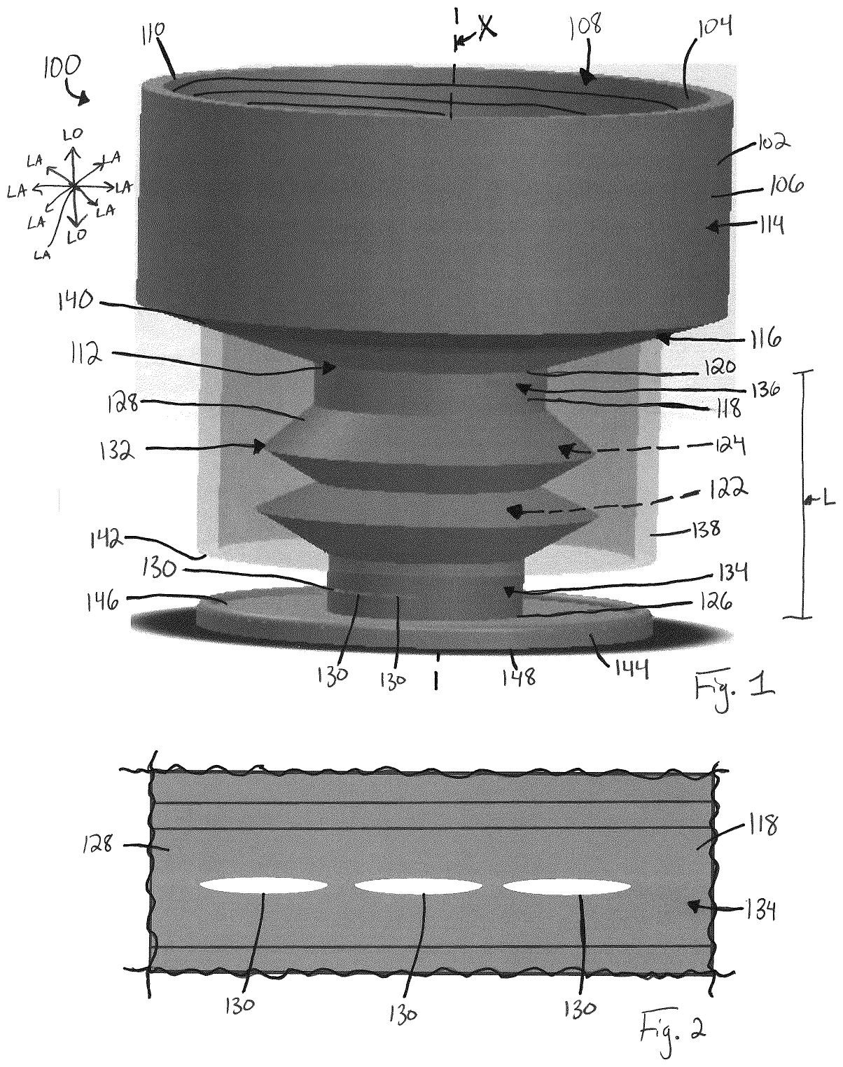

FIG. 1 depicts an example nozzle 100 designed in accordance with the present disclosure. The nozzle 100 may include an adapter 102 having inner and outer adapter surfaces 104, 106. The inner adapter surface 104 may define an inner adapter chamber 108 longitudinally extending between first and second adapter openings 110, 112. The term “longitudinal” is used herein to indicate a substantially vertical direction, in the orientation of FIG. 1, with the “longitudinal” direction being indicated as “LO” in FIG. 1.

The adapter 102 may be defined by longitudinally adjacent first and second adapter portions 114, 116. The first adapter opening 110 may be located at the first adapter portion 114, while the second adapter opening 112 may be located at the second adapter portion 116. As shown in FIG. 1, the first adapter portion 114 may be substantially shaped as a cylinder, and the second adapter portion 116 may be substantially shaped as a truncated cone. However, the first adapter portion 114, the second adapter portion 116, and/or the adapter 102 (as a whole) may have any desired shape for any given use environment.

A nozzle head 118 is connected to and extends longitudinally along a longitudinal axis X of the nozzle 100 from the outer adapter surface 106. In particular, the nozzle head 118 includes a first head end 120 connected to the outer adapter surface 106 at the second adapter portion 116. The first head end 120 is positioned on the outer adapter surface 106 such that an inner head chamber 122 of the nozzle head 118 is in fluid communication with the adapter chamber 108 via the second adapter opening 112. The head chamber 122 is defined by an inner head surface 124 of the nozzle head 118 and is configured to selectively hold a fluid (e.g., a therapeutic ophthalmic liquid, which may be a therapeutic ophthalmic fluid having a viscosity of about 1 cps to about 200 cps). The nozzle head 118 may be substantially hollow so that the head chamber 122 extends longitudinally between the first head end 120 and a longitudinally opposite second head end 126 of the nozzle head 118.

The nozzle head 118 also includes a head body 128 separating and extending substantially longitudinally between the first and second head ends 120, 126. The head body 128 may include one or more nozzle openings or slits 130 (as shown here by way of example, three nozzle openings or slits 130) through which fluid is configured to be selectively delivered from the head chamber 122 to a target site during use. As shown in FIG. 1, the nozzle openings 130 are adjacent to the second head end 126 and extend laterally through the head body 128, though the nozzle 100 may be configured such that the nozzle openings 130 are at any other desired longitudinal position along head body 128. The term “lateral” is used herein to indicate a direction substantially perpendicular to the “longitudinal” direction, with the “lateral” direction being indicated as “LA” in FIG. 1. The nozzle openings 130 may be spaced evenly along a circumference of the head body 128. A portion of the head body 128 may also define a bellows 132 configured to be selectively longitudinally compressed and decompressed under a striking force manually applied by a user, a striking force mechanically applied by a mechanism (such as, e.g., a solenoid-type actuator), and/or an inherent elastic-deformation bias of the bellows 132 itself.

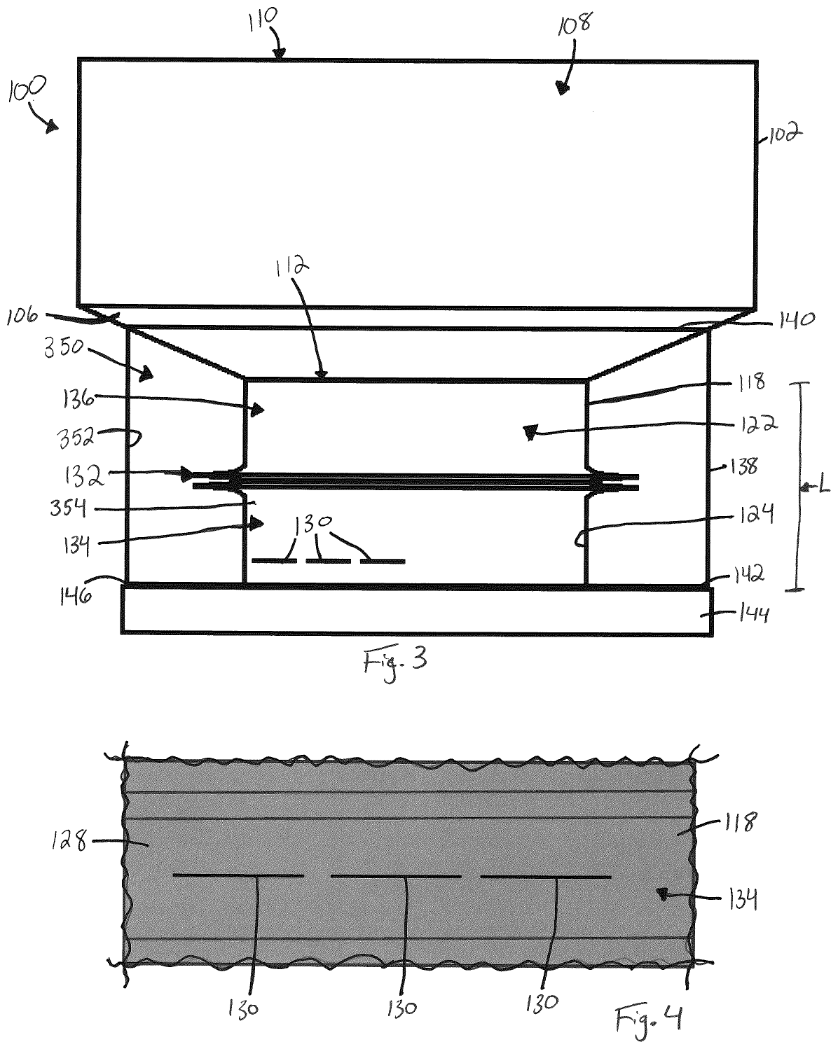

As shown in FIGS. 1-2, the nozzle head 118 has an opened condition in which the nozzle openings 130 are at least partially open to permit fluid flow therethrough. The nozzle head 118 is selectively deflectable between the opened condition (FIGS. 1-2) and a closed condition (FIGS. 3-4). In particular, the nozzle head 118 may be longitudinally compressed from the opened condition toward the closed condition in response to an external longitudinal compressive force applied to the nozzle head 118. At least a portion of the nozzle head's ability to compress is provided by the bellows 132, which compresses in the longitudinal direction in response to the applied external compressive force.

The nozzle head's ability to compress may also be at least partially provided via the natural properties of a material selected to form the nozzle head 118. For example, a second head portion 134 of the head body 128 (i.e., a portion of the head body 128 longitudinally between the bellows 132 and the second head end 126) may be at least partially formed from a deformable material. In such case, the external compressive force not only causes the bellows 132 to compress, the force also causes at least the second head portion 134 to compress. Any other portion of the head body 128, such as, e.g., a first head portion 136 (i.e., a portion of the head body 128 longitudinally between the bellows 132 and the first head end 120) and/or the bellows 132 portion, may be at least partially formed from a deformable material so as to compress under the applied longitudinal compressive force. Therefore, because of the bellows 132 and/or the deformable material, the nozzle head 118 is permitted to longitudinally compress under an applied longitudinal compressive force from the opened condition to the closed condition.

As shown in FIGS. 1-4, the compression of the second head portion 134 urges the nozzle openings 130 to at least partially close as the nozzle head 118 is compressed to the closed condition. Therefore, not only is a longitudinal length L of the nozzle head 118 reduced when the nozzle head 118 is in the closed condition, the nozzle openings 130 are also at least partially closed. The closure (or at least partial closure) of the nozzle openings 130 restricts and/or prevents fluid from flowing through the nozzle openings 130.

As shown in FIG. 2, each of the nozzle openings 130, when in the opened condition, may have a substantially elliptical shape with a longitudinal opening length that is significantly smaller than a lateral opening width. The shape of the nozzle openings 130 are selected so that the at least partial closure of the nozzle openings is relatively easy. The nozzle openings 130 may have any shape(s) as desired to achieve the opening/closing properties described herein.

After reaching the closed condition, the nozzle head 118 may be selectively deflected (e.g., expanded/decompressed) to the opened condition via an external longitudinal decompressive force (i.e., an external force that is opposite to that of the external longitudinal compressive force) applied to the nozzle head 118. In addition to or instead of the external longitudinal decompressive force, the deflection of the nozzle head 118 back to the opened condition may additionally or instead be accomplished via the natural properties of the nozzle head 118 material. For example, in addition to being at least partially formed from a deformable material, the nozzle head 118 may also be at least partially formed from an elastic material. In such case, upon removal of the external compressive force, the elastic properties of the nozzle head 118 may urge the nozzle head 118 to at least partially expand from the closed condition toward the opened condition.

As shown in FIGS. 1-4, the nozzle 100 includes a shielding wall 138 having a first shielding end 140 connected to the outer adapter surface 106 at the second adapter portion 116. The shielding wall 138 extends along the longitudinal axis from the adapter 102 to a second shielding end 142. The shielding wall 138 is coaxial with the nozzle head 118 so that the shielding wall 138 at least partially encircles a portion of the nozzle head 118. When the nozzle head 118 is in the opened condition (FIG. 1), the shielding wall 138 does not “cover” the nozzle openings 130 because the nozzle openings 130 are longitudinally offset from shielding wall 138. However, in the closed condition (FIG. 3), the nozzle openings 130 are longitudinally aligned with the shielding wall 138 and are thus “covered” by the shielding wall 138. The shielding wall 138 (or at least a portion thereof) may be at least semi-transparent or opaque.

The nozzle 100 also includes a closure plate 144 having longitudinally opposite first and second plate ends 146, 148. The first plate end 146 is connected to the second head end 126. When the nozzle head 118 is in the opened condition (FIG. 1), the closure plate 144 is spaced from the shielding wall 138. However, when the nozzle head 118 is in the closed condition (FIG. 3), the first plate end 146 engages the second shielding end 142. As shown in FIG. 3, a moisture chamber 350 is collectively formed and defined by an interior surface 352 of the shielding wall 138, the outer adapter surface 106, the first plate end 146, and an outer nozzle surface 354 of the nozzle head 118 when the nozzle head 118 is in the closed condition.

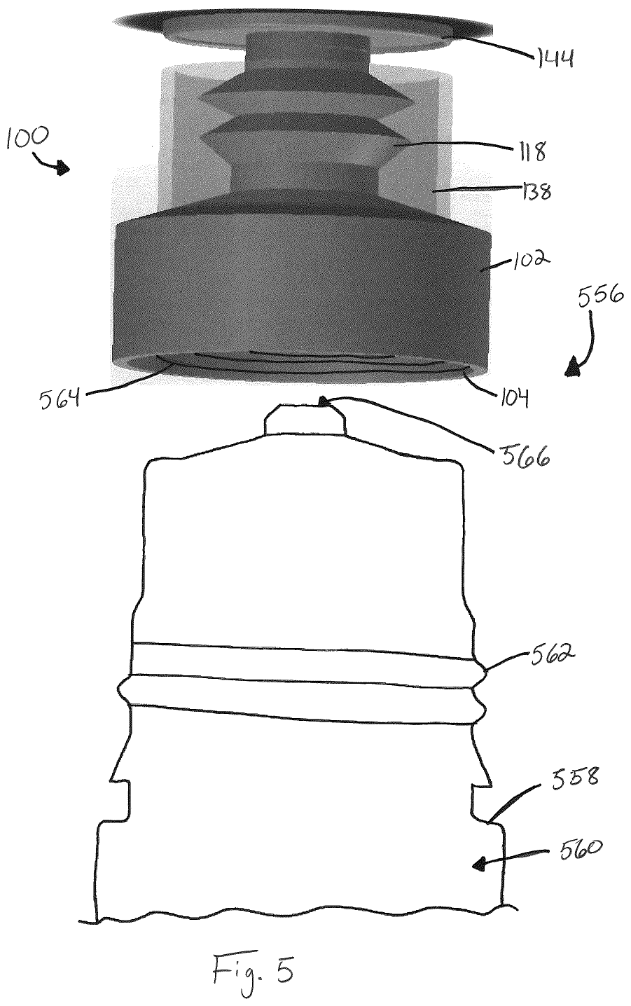

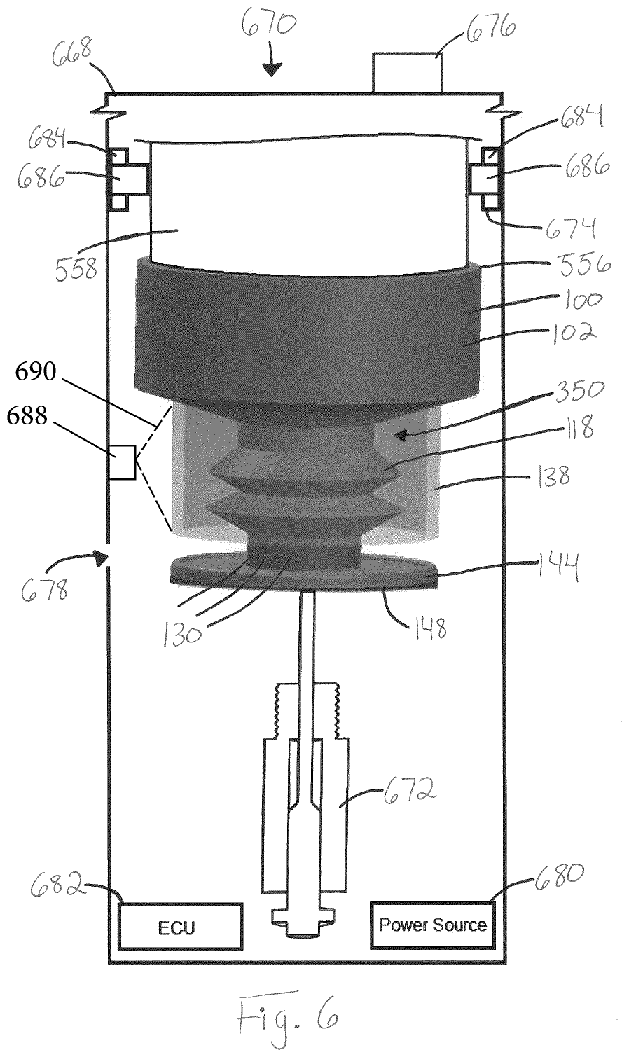

As shown in FIG. 5, the nozzle 100 may be a part of a cartridge 556 that also includes a container 558. The container 558 has an inner container chamber 560 configured to accommodate a fluid (e.g., a therapeutic ophthalmic liquid, which may be a therapeutic ophthalmic fluid having a viscosity of about 1 cps to about 200 cps). The container 558 may also include external threads 562 that are configured to selectively mate with internal threads 564 on the inner adapter surface 104 to couple and secure the nozzle head 118 to the container 558. When secured to the container 558, the head chamber 122 is in fluid communication (e.g., selective fluid communication) with the container chamber 560 such that the fluid selectively flows out from the container chamber 560 via a container opening 566 and into the head chamber 122.

The container 558 may be a multi-dose preservative-free (MDPF) bottle having a one-way liquid valve and a separate filtered air port for pressure equalization. An example of such an MDPF bottle is discussed in U.S. Pat. No. 9,345,616, issued 24 May 2016 to Grevin at al. and titled “LIQUID DISPENSING DEVICE EQUIPPED WITH AN AIR DUCT”, the subject matter of which is incorporated herein by reference in its entirety. The container 558, however, may be any desired holder/device configured to accommodate a fluid. Further, although the nozzle 100 is described as being “screwed” onto the container 558 via the internal threads 564, the nozzle 100 may be coupled and/or secured to the container 558 in any suitable manner. Other attachment features (e.g., press-fit interference nubs and/or anti-twist off features) may be provided in addition to the internal/external threads 564/562 to further secure the nozzle 100 to the container 558. The attachment of the nozzle 100 to the container 558 (whether it be via the threads 562/562 and/or any other attachment feature) may be such that a liquid and bacterial seal is formed between the nozzle 100 and the container 558. This seal may be formed in a sterile manufacturing environment prior to use. The nozzle 100, however, could also be configured such that the nozzle 100 and the container 558 are integrally or monolithically formed as a single piece.

In use, the cartridge 556 may be provided to a user. The cartridge 556 may be provided in in a fully-assembled state, or in a pre-assembled state in which the user then couples the nozzle 100 to the container 558. As shown in FIG. 6, the cartridge 556 may be inserted into an applicator 668 that is configured to accommodate the cartridge 556. The cartridge 556 and the applicator 668 thus collectively define a fluid delivery device 670 (e.g., a non-gravitational fluid delivery device). The fluid delivery device 670 may be provided to the user in a fully-assembled state with the cartridge 556 already loaded into the applicator 668, or may be provided in a pre-assembled state in which the user then loads the cartridge 556 into the applicator 668.

The applicator 668 includes an actuator 672, a dispensing mechanism 674, at least one trigger 676 operatively connected to the actuator 672 and the dispensing mechanism 674, and an applicator opening 678 through which fluid may be selectively ejected from the applicator 668 and the fluid delivery device 670. The applicator 668 may be similar to, be a modified version of, or contain elements (e.g., the sensors) of the applicator disclosed in U.S. Pat. No. 11,510,809, issued 29 Nov. 2022 to Stowe and titled “NON-GRAVITATIONAL FLUID DELIVERY DEVICE FOR OPHTHALMIC APPLICATIONS”, the subject matter of which is incorporated herein by reference in its entirety, the applicator disclosed in U.S. Pat. No. 11,707,381, issued 25 Jun. 2023 to Stowe and titled “OCULAR PHARMACEUTICAL APPLICATOR WITH LIGHT-ASSISTED ALIGNMENT AND AIMING”, the subject matter of which is incorporated herein by reference in its entirety, or any other suitable applicator.

The applicator 668 may also include a power source 680 and an electronic control unit (“ECU”) 682. The power source 680 may be a rechargeable battery, such as a small LiPo coin cell battery. The ECU 682 may be operably coupled to the power source 680, the actuator 672, the dispensing mechanism 674, and the trigger 676. Therefore, at least one of the power source 680, the actuator 672, the dispensing mechanism 674, and the trigger 676 may be operably connected to at least one other of the power source 680, the actuator 672, the dispensing mechanism 674, and the trigger 676 via the ECU 682. The actuator 672 may be bi-stable solenoid (as is shown in FIG. 6), a linear actuator, or any other suitable actuator.

The dispensing mechanism 674 may include a motor portion 684, which comprises, includes, or houses a motor (e.g., an electric motor), and a force applying portion 686 that is moveable via the motor relative to the motor portion 684. Actuation of the dispensing mechanism 674 (e.g., via actuation of the motor) causes the force applying portion 686 to move toward and apply a force to the container 558 (e.g., a lateral force that “squeezes” the container 558). The applied force is configured to urge a predetermined amount of the fluid from the container 558 longitudinally into the head chamber 122. The dispensing mechanism 674 (or the ECU 675) may be calibrated by the user or a medical professional to select the amount of fluid that is expelled into the head chamber 122 by the dispensing mechanism 674.

When the cartridge 556 is loaded in the applicator 668, the nozzle head 118 may be maintained in the closed condition (e.g., via the actuator 672) so that the moisture chamber 350 is formed. By holding the nozzle head 118 in the closed condition, the nozzle openings 130 are fully or at least partially sealed in the moisture chamber 350, and thus are at least partially protected from outside contaminants.

When desired, the user may align the applicator opening 678 with a target site (e.g., an eye of the user), and then manipulate the trigger 676 (e.g., by depressing the trigger 676) to activate the actuator 672 and the dispensing mechanism 674. For example, the manipulated trigger 676 may send an activation request signal to the ECU 675, which then sends activation signals to each of the actuator 672 and the dispensing mechanism 674. The activated dispensing mechanism 674 applies a force to the container 558 that urges a predetermined amount of the fluid from the container 558 longitudinally into the head chamber 122. Prior to, as, or after the dispensing mechanism 674 dispenses the fluid from the container 558, the activated actuator 672 moves to a loaded position (FIG. 6). As the actuator 672 moves to the loaded position, the nozzle head 118 expands to the opened condition and the nozzle openings 130 are at least partially opened.

The actuator 672 moving to the loaded position may directly and/or indirectly cause the nozzle head 118 to expand to the opened condition. For example, the actuator 672 may be connected to the closure plate 144, such as to the second plate end 148. This may be a direct connection (as is shown in FIG. 6), or an indirect connection via one or more intermediate components. In such a configuration, as the actuator 672 moves to the loaded position, the actuator 672 applies a longitudinal decompressive force to the closure plate 144 that pulls and/or urges the closure plate 144 away from the shielding wall 138, which in turn expands/decompresses the nozzle head 118 to the opened condition. The actuator 672 thus may directly cause the nozzle head 118 to expand to the opened condition.

The natural properties of the nozzle head 118, when formed from an elastic and deformable material, may also directly cause the nozzle head 118 to at least partially expand to the opened condition. For example, prior to use, the actuator 672 may apply a longitudinal compressive force to the closure plate 144 that retains the nozzle head 118 in the closed position against an elastic bias of the nozzle head 118. When the actuator 672 is activated, the actuator 672 may simply move to the loaded position without pulling or urging the closure plate 144 away from the adapter 672. However, as the actuator 672 moves to the loaded position, the longitudinal compressive force is at least partially alleviated so that the internal bias of the nozzle head 118 is permitted to decompress and expand the nozzle head 118 in the longitudinal direction to the opened condition. Therefore, the activation of the actuator 672 may indirectly cause the nozzle head 118 to transition to the opened condition, while the internal bias of the nozzle head 118 directly causes this transition. The fluid delivery device 670, however, may be configured such that the actuator 672 and the elastic nature of the nozzle head 118 may each at least partially directly cause the nozzle head 118 to transition to the opened condition in their respective manners described above.

Although the fluid has been described as being urged into the head chamber 122 via the force provided by the dispensing mechanism 674, the fluid may also or instead be urged into the head chamber 122 via negative pressure generated in the head chamber 122 as the nozzle head 118 moves from the closed condition to the opened condition. In other words, the nozzle head 118 moving to the opened condition may be configured to generate enough negative pressure in the head chamber/container 122/558 to suction fluid from the container chamber 560 into the head chamber 122.

Once the nozzle head 118 reaches the opened condition, the nozzle openings 130 are at least partially opened and aligned with the applicator opening 678. The actuator 672 then moves from the loaded position to a striking position. This movement of the actuator 672 may be in response to the same trigger 676 manipulation as described above, or in response to a second trigger 676 manipulation. The actuator 672 applies a longitudinal compressive force to the closure plate 144 as the actuator 672 moves to the striking position. The longitudinal compressive force responsively urges the closure plate 144 toward the shielding wall 138, and thus urges the nozzle head 118 to compress to the closed condition (optionally against the internal bias of the nozzle head 118). The compression of the nozzle head 118 pressurizes the head chamber 122 to such a degree that the fluid in the head chamber 122 is ejected in the lateral direction from the head chamber 122 and to the user's eye through the nozzle and applicator openings 130, 678. The flow of the fluid thus changes direction (e.g., about a 90 degree change in direction, in some use environments) within the nozzle head 118 from a longitudinal or “vertical” flow (from the container chamber 560 into and along at least a portion of the head chamber 122) to a lateral or “horizontal” flow (from the head chamber through the nozzle openings 130). The nozzle 100 thus converts what would have been a substantially “vertical” delivery of fluid from the container 558 into a substantially “horizontal” delivery of fluid from the fluid delivery device 670. The actuator 672 may be calibrated by the user or a medical professional to provide a predetermined longitudinal compressive force so that the same fluid delivery device 670 may be used for a variety of fluids having varying levels of viscosity.

The nozzle openings 130 at least partially close as the nozzle head 118 compresses to the closed condition at least partially ends the ejection of fluid. Therefore, the closing of the nozzle openings 130 may cause a small terminal portion of the ejecting fluid to be prevented from leaving the nozzle 100. Such a cut-off helps provide a consistent dosage (in terms of amount and droplet/stream size/shape) of fluid to the user during each use.

The moisture chamber 350 is also formed/closed as the closure plate 144 is moved longitudinally into engagement with the shielding wall 138. Sealing the nozzle openings 130 in the moisture chamber 350 not only helps protect the nozzle openings 130 from outside contaminants, it also at least partially prevents air from drying any excess fluid in or adjacent the nozzle openings 130. In other words, the closed moisture chamber 350 helps support a moist and humid environment within the moisture chamber 350. Because of the moisture and humidity, excess fluid cut-off (from the tailing portion) or otherwise left in or adjacent to the nozzle openings 130 is at least partially prevented from drying and potentially causing obstructions along the flow path through the nozzle openings 130. Furthermore, because the nozzle openings 130 are at least partially sealed in the moisture chamber 350, the nozzle openings 130 do not have to fully close. The nozzle openings 130 may instead close just enough that fluidic impedance prevents fluid from escaping toward the end of the ejection process.

As shown in FIG. 6, the applicator 668 (or any other portion of the fluid delivery device 670) may include at least one sterilizing light emitting diode (LED) 688 of violet or ultraviolet light. The violet or ultraviolet light emitted by the LED may have a wavelength of about 200 nm to about 400 nm. The sterilizing LED 688 may be positioned in the applicator 668 such that the at least one of the moisture chamber 350 (or at least a portion of the moisture chamber 350) and the nozzle openings 130 are selectively exposed to an LED light cone 690 emitted from the sterilizing LED 688. The sterilizing LED 688 may be powered via the power source 680 and controlled via the ECU 682 so as to be turned on and off by the ECU 682. For example, the ECU 682 may be programmed having a disinfection routine such that excess fluid in the moisture chamber and/or in or adjacent to at least one of the nozzle openings 130 is at least partially disinfected on a periodic basis via actuation of the sterilizing LED 688. The disinfection routine may be designed such that the sterilizing LED 688 is actuated before a dispensing event, after a dispensing event, when the moisture chamber 350 is closed, and/or when the moisture chamber 350 is opened (i.e., when the closure plate 144 is spaced longitudinally from the shielding wall 138).

Although the sterilizing LED 688 is shown as being external to the moisture chamber 350, the sterilizing LED 688 may be positioned within the moisture chamber 350. For example, the sterilizing LED 688 may be in the moisture chamber 350 and connected to at least one of the adapter 102, the nozzle head 118, the shielding wall 138, and the closure plate 144.

The fluid delivery device 670 may be a single- or multi-use device. In the case of multi-use, the user may utilize the device 670 in the manner as described above until the container 558 is empty. Once empty, the cartridge 556 may be removed and replaced. The container 558 may be configured to be a single-use or a refillable container 558. The nozzle 100 may also be configured for a single or multiple uses. In the case of multi-use, the nozzle 100 may be removed from an at least partially empty container 558 and then re-coupled to the container 558 after it has been refilled, or coupled to another fluid-holding container 558.

Although the cartridge 556 has been described as being a part of a fluid delivery device 670 that also includes the applicator 668, the cartridge 556 on its own can define a fluid delivery device 670. In other words, in certain use cases, the applicator 668 may not be provided as a part of the fluid delivery device 670, and the user may instead function as the “applicator.” In such case, the user may selectively manipulate cartridge 556 to dispense fluid from the container chamber 560 into the head chamber 122 (e.g., by “squeezing” the container 558), move the nozzle head 118 between the opened and closed conditions (e.g., by longitudinally moving closure plate 144), and eject fluid from the head chamber 122 as desired (e.g., by providing a longitudinal compressive force to the nozzle head 118 via closure plate 144).

Further, instead of the applicator 668 including a dispensing mechanism 674, the user may apply a force to the container 558 that responsively causes fluid to be urged from the container 558 into the head chamber 122.

It is contemplated the nozzle 100 and/or the fluid delivery device 670 may also include a removable nozzle cap for the nozzle 100. When coupled to the nozzle 100, the cap may be configured to retain the nozzle head 118 in the closed condition and the closure plate 144 engaged to the shielding wall 138 via a longitudinal compressive force. The nozzle cap may be selectively removed from the remainder of the nozzle 100 prior to use.

While aspects of this disclosure have been particularly shown and described with reference to the example aspects above, it will be understood by those of ordinary skill in the art that various additional aspects may be contemplated. For example, the specific methods described above for using the apparatus are merely illustrative; one of ordinary skill in the art could readily determine any number of tools, sequences of steps, or other means/options for placing the above-described apparatus, or components thereof, into positions substantively similar to those shown and described herein. In an effort to maintain clarity in the Figures, certain ones of duplicative components shown have not been specifically numbered, but one of ordinary skill in the art will realize, based upon the components that were numbered, the element numbers which should be associated with the unnumbered components; no differentiation between similar components is intended or implied solely by the presence or absence of an element number in the Figures. Any of the described structures and components could be integrally formed as a single unitary or monolithic piece or made up of separate sub-components, with either of these formations involving any suitable stock or bespoke components and/or any suitable material or combinations of materials. Any of the described structures and components could be disposable or reusable as desired for a particular use environment. Any component could be provided with a user-perceptible marking to indicate a material, configuration, at least one dimension, or the like pertaining to that component, the user-perceptible marking potentially aiding a user in selecting one component from an array of similar components for a particular use environment. A “predetermined” status may be determined at any time before the structures being manipulated actually reach that status, the “predetermination” being made as late as immediately before the structure achieves the predetermined status. The term “substantially” is used herein to indicate a quality that is largely, but not necessarily wholly, that which is specified—a “substantial” quality admits of the potential for some relatively minor inclusion of a non-quality item. Though certain components described herein are shown as having specific geometric shapes, all structures of this disclosure may have any suitable shapes, sizes, configurations, relative relationships, cross-sectional areas, or any other physical characteristics as desirable for a particular application. Any structures or features described with reference to one aspect or configuration could be provided, singly or in combination with other structures or features, to any other aspect or configuration, as it would be impractical to describe each of the aspects and configurations discussed herein as having all of the options discussed with respect to all of the other aspects and configurations. A device or method incorporating any of these features should be understood to fall under the scope of this disclosure as determined based upon the claims below and any equivalents thereof.

Other aspects, objects, and advantages may be obtained from a study of the drawings, the disclosure, and the appended claims.

I claim:

1. A nozzle for a fluid delivery device, the nozzle comprising:
a nozzle head defining an inner head chamber for a fluid, the nozzle head having at least one nozzle opening through which fluid is configured to be selectively delivered from the head chamber to a target site during use of the device, the nozzle head being at least partially formed from a deformable material, the nozzle head being selectively deflectable between an opened condition in which the at least one nozzle opening is at least partially open to permit fluid flow therethrough and a closed condition in which the at least one nozzle opening is at least partially closed to restrict fluid flow therethrough.

2. The nozzle of claim 1, wherein the nozzle head is at least partially formed from a deformable and elastomeric material.

3. The nozzle of claim 1, wherein the nozzle head includes a first head end, a second head end, and a head body separating and extending longitudinally between the first and second head ends, a portion of the head body defining a bellows configured to be selectively compressed to urge the fluid through the at least one nozzle opening.

4. The nozzle of claim 1, wherein, as the nozzle head transitions from the opened condition to the closed condition, the nozzle head becomes at least partially longitudinally compressed and the at least one nozzle opening is at least partially closed.

5. The nozzle of claim 4, wherein as the nozzle head transitions from the closed condition to the opened condition, the nozzle head at least partially longitudinally expands to and open the at least one nozzle opening at least partially opens.

6. The nozzle of claim 1, further comprising a shielding wall encircling at least a portion of the nozzle head.

7. The nozzle of claim 6, wherein a portion of the shielding wall is at least partially longitudinally aligned with the at least one nozzle opening when the nozzle head is in the closed condition, the shielding wall being at least partially longitudinally offset from the at least one nozzle opening when the nozzle head is in the opened condition.

8. The nozzle of claim 6, wherein a moisture chamber is defined between an outer surface of the nozzle head and an interior surface of the shielding wall when the nozzle head is in the closed condition.

9. The nozzle of claim 8, wherein the nozzle head includes a first head end, a second head end, and a head body separating and extending longitudinally between the first and second head ends, the at least one nozzle opening being adjacent the second head end, the nozzle further comprising a closure plate connected to the second head end, the closure plate engaging the shielding wall when the nozzle head is in the closed condition to at least partially define the moisture chamber, the closure plate being spaced longitudinally from the shielding wall when the nozzle head is in the opened condition.

10. The nozzle of claim 1, further comprising an adapter for selectively securing the nozzle to a fluid-holding container, the adapter being connected to the nozzle head.

11. The nozzle of claim 10, wherein the nozzle head includes a first head end connected to the adapter, a second head end, and a head body separating and extending longitudinally between the first and second head ends, the at least one nozzle opening being adjacent the second head end.

12. The nozzle of claim 11, further comprising:
a shielding wall longitudinally extending from the adapter and encircling at least a portion of the nozzle head; and
a closure plate connected to the second head end, the closure plate engaging the shielding wall when the nozzle head is in the closed condition so that the adapter, shielding wall, and closure plate collectively define a moisture chamber, the closure plate being spaced longitudinally from the shielding wall when the nozzle head is in the opened condition.

13. The nozzle of claim 1, wherein the at least one nozzle opening comprises a plurality of nozzle openings.

14. The nozzle of claim 1, wherein the fluid is a therapeutic ophthalmic fluid and the target site is a user's eye.

15. A fluid delivery device for delivering fluid to an eye of a user, comprising:
a cartridge including a container and the nozzle of claim 1 coupled to the container, the container having an inner container chamber configured to accommodate a fluid, the head chamber being in fluid communication with the container chamber such that the fluid selectively flows from the container chamber to the head chamber; and
an applicator configured to accommodate the cartridge, the applicator including an actuator for selectively manipulating the nozzle head between the opened condition and the closed condition.

16. The fluid delivery device of claim 15, wherein the fluid delivery device is a non-gravitational fluid delivery device.

17. The fluid delivery device of claim 15, wherein actuator compresses the nozzle head toward the closed condition to cause the fluid to eject from the holding chamber via the at least one nozzle opening, the at least one nozzle opening at least partially closing as the nozzle head is compressed.

18. The fluid delivery device of claim 15, wherein upon actuation of the application, the actuator:
moves to a loaded position, the nozzle head decompressing to the opened condition as the actuator moves to the loaded position; and subsequently
moves from the loaded position to a striking position, the nozzle head compressing to the closed condition as the actuator moves to the striking position, the fluid from the head chamber being ejected from the head chamber as the nozzle head compresses.

19. A method of delivering fluid to a user's eye, the method comprising:
providing the fluid delivery device of claim 15;
inserting the cartridge into the applicator;
aligning an applicator opening of the applicator with the user's eye;
activating the actuator to manipulate the nozzle head from the opened condition to the closed condition; and
delivering fluid from the head chamber, through the nozzle and applicator openings, and to the user's eye as the nozzle head is manipulated from the opened condition to the closed condition.
